(19) United States
(12) Patent Application Publication
(10) Pub. No.: US 2021/0236687 A1
(43) Pub. Date: Aug. 5, 2021

(54) CANNABINOID-BASED ORAL HEMOSTATIC COMPOSITIONS

(71) Applicant: Pac-Dent, Inc., Brea, CA (US)

(72) Inventors: Daniel Wang, Brea, CA (US); Xiao Yang, La Habra, CA (US); Heng Sun, Chino, CA (US)

(73) Assignee: Pac-Dent, Inc., Brea, CA (US)

(21) Appl. No.: 16/779,419

(22) Filed: Jan. 31, 2020

(51) Int. Cl. A61L 26/00

(52) U.S. Cl. CPC A61L 26/0028; A61L 26/0004; A61L 2300/10; A61L 2300/20; A61L 26/0066

(57) ABSTRACT

The present invention relates to a wound management composition consisting of full spectrum blend of active cannabinoids and at least one hemostatic agent including astringent-class hemostats, active-class hemostats, and active-mechanical-class hemostats. Further, the composition may also include at least chemical penetration enhancer that amplifies the absorption of cannabinoids, at least one local drug to prolong the pharmacologically active time of cannabinoids, at least one antibacterial agent, and at least one antifungal agent. Methods of delivery of the wound management composition and kits are also disclosed.

### BACKGROUND OF THE INVENTION

### Field of the Invention

The present invention relates generally to wound management systems, and more specifically to oral hemostasis formulations containing a full spectrum blend of active cannabinoids and at least one component including hemostatic agents, as well as compositions/kits containing such formulations and methods of using said formulations.

### Background Information

Bleeding (or hemorrhaging) in a clinical environment is an inescapable aspect of modern medicine. It is the result of any surgical operation that disrupts and damages blood vessels. The consequences of excessive bleeding are significant and must be addressed accordingly. It can lead to infection, hematoma (bruising), prolonged wound healing, dehiscence (wound separation), and even necrosis. There exists a variety of solutions to control bleeding during any surgical procedure and can be broadly separated into two categories: hemostatic tools such as forceps and clamps that manually close off a bleeding blood vessel and hemostatic agents such as aluminum sulfate and porcine gelatin that utilize various mechanisms to induce hemostasis. These latter hemostatic agents may simply be referred to as hemostats, henceforth. These hemostats can be further divided into several classes: astringent, active, mechanical, and active-mechanical combinations.

Astringent hemostats such aluminum sulfate, aluminum chloride, ferric sulfate, and zinc chloride are commonly used hemostatic agents in the oral environment. They are applied topically and effect localized hemostasis by coagulating and agglutinating blood proteins to mechanically form a barrier closing off ruptured vessels upon contact with blood. These hemostats are commonly used in dentistry for their ease of use and short onset of action (e.g., within 1 to 3 minutes).

Epinephrine is another frequently used dental hemostat. While it does not induce any coagulation, it is a vasoconstrictor that causes hemostasis by reducing the size of blood vessels. Epinephrine can be impregnated in gingival retraction cords, which push the gumline away from the tooth to give the dentist a more clear view of the tooth. This often results in mild to moderate bleeding, needing the use of a hemostat. However, there is concern over its use as a hemostat due to well documented side effects of elevated blood pressure and heart rate after use.

The principal ingredient of active hemostats is thrombin. Thrombin is the most important constituent of the body's blood clotting process. It is a proteolytic enzyme that converts fibrinogen, a soluble glycoprotein in blood, into fibrin, a fibrous protein that attracts platelets to form a clot over a wound entrance (see FIG. 1). Thrombin-based hemostats are further divided into three subclasses, depending on where the thrombin is sourced. The oldest subclass dates back to the 1970s and uses bovine thrombin, which may cause adverse reactions due to the body's antibodies potentially attacking the bovine thrombin. EVITHROM™ does not have the same problems as bovine thrombin and uses human pooled thrombin. Even though it is carefully screened and tested, EVITHROM™ still carries a risk of viral transmission. RECOTHROM™ uses recombinant thrombin and does not contain human or bovine thrombin. RECOTHROM™ was FDA approved in 2008.

Further, fibrin sealant hemostats such as EVICEL™ and TISSEEL™ contain both thrombin and fibrinogen. They are used when both hemostasis and sealing are desired. Fibrinogen is combined with thrombin to cause immediate fibrin creation and results in clot initiation. These products are kept in frozen storage and need to be thawed before use. Fibrin sealants are less often used due to their relatively high cost.

The final class of hemostatic agents is a combination of active and mechanical hemostats. These products provide both an active and mechanical mechanism in achieving hemostasis. Flowable hemostats such as FLOSEAL™ and SURGIFLO™ combine gelatin with thrombin. The gelatin expands upon contact with blood to provide a physical matrix for clot formation while the thrombin initiates the actual clot formation. Flowables are used when application is required for hard to reach areas. Like sealants, these hemostats are relatively expensive and less frequently used.

Although there is no doubt in the efficacy of hemostatic agents in inducing hemostasis, they do not provide any additional therapeutic or physiological benefits that are desired for wound healing. These benefits include anti-bacterial, anti-inflammatory, and pain-relieving benefits, which can be added to topical hemostatic agents via inclusion of additional chemical constituents.

As Cannabis decriminalization and legalization spread across the United States in the last decade, there has been a wealth of new medical research concerning Cannabis and its pharmacological properties. Of note are cannabinoids, various chemical compounds found within the cannabis plant that trigger a variety of different physiological effects within the human body.

Cannabinoids refer to various non-synthetic phytocannabinoids found in the Cannabis botanical genus. Although over 200 different cannabinoids have been discovered within the Cannabis plant, only a few have had significant research done on them. Of this latter subgroup, tetrahydrocannabinol (THC), cannabidiol (CBD), cannabinol (CBN), cannabichromene (CBC), and cannabigerol (CBG) stand out for their medical significance. These aforementioned cannabinoids have shown anti-bacterial, anti-inflammatory, and pain-relieving properties in multiple peer-reviewed studies. A brief literature review on the subject matter will be discussed below.

Appendino et al. (J Natural Products (2008) 71 (8):1427-1430) discovered that all five of the aforementioned cannabinoids displayed greater antibacterial activity against a variety of common methicillin-resistant Staphylococcus aureus strains (MRSA) than the standard antibiotic treatment for the corresponding strain. Briefly, MRSA refers to strains of the common Staphylococcus aureus that have acquired multiple drug resistance through natural selection and horizontal gene transfer. MRSA is the cause of several difficult to treat infections in humans. Although MRSA infections can occur in many areas of the body, it is most commonly localized on the skin.

Petrosino et al. (J Pharm Exptl Thera (2018) 365(3):652-663) found that CBD showed potent anti-inflammatory properties in an in vitro model of allergic contact dermatitis (ACD), a skin inflammatory response that occurs after dermal contact with an allergen, by inhibiting the production of the MCP-2 chemokine and other proinflammatory cytokines, whose job it is to activate immune cells and induce inflammation. Other cannabinoids such as CBG, CBC, and cannabidivarin (CBDV) were also shown to have some anti-inflammatory benefits, but required a much higher concentration dosage and to a lesser extent compared to CBD.

Toth et al. (Molecules (2019) 24(5):918) presented a literature survey on the relationship between the endocannabinoid system and skin. The authors reported that while CBD is commonly known as a CB1 antagonist, it can also act as a context-dependent CB agonist. This allows CBD to signal though the endocannabinoid system to maintain skin homeostasis and aid barrier formation and regeneration. CBD was shown in multiple clinical studies in vivo and in vitro to improve skin conditions varying from dry skin to atopic dermatitis such as eczema, psoriasis, and scleroderma.

THC's anti-inflammatory properties have been well documented in the past few decades. It has been shown to lower inflammation by inhibiting the synthesis of PGE-2 and stimulating the synthesis of lipooxygenase. THC's anti-inflammatory potency is reported to be twenty times that of aspirin and twice that of hydrocortisone. (Evans, Planta Med. (1991) 57(7):S60-67). However, in contrast to the aforementioned drugs, THC achieves this without any cyclooxygenase (COX) inhibition, which relieves any concerns of potential gastrointestinal ulcers and bleeding.

A meta-study by Russo (Ther Clin Risk Manag (2008) 4(1):245-259) on cannabinoids and pain found that all five of the aforementioned cannabinoids except cannabinol were effective analgesics for both acute and chronic pain with minimal adverse effects. (Id). Specifically, orally ingested cannabinoids were shown to lower daily pain and reduce acute hyperalgesia in a variety of conditions when compared to the placebo treatment in multiple clinical trials. This is believed to be achieved by stimulation of the CB receptor.

Although cannabinoids are lipid-soluble and may be absorbed through the skin, its transdermal absorption is significantly less that of oral ingestion or inhalation. The primary physical barrier to transdermal drug delivery is the stratum corneum, the outermost layer of the skin comprising of about 20 layers of flattened cells. Accordingly, there exists a need to improve upon the topical absorption of cannabinoids in the art. Terpenes are a large and diverse category of significantly odorous organic compounds, which are mainly found in plants. Terpenes such as limonene, linalool, pinene and the like were found to act as chemical penetration enhancers (CPE) for cannabinoids. (Bruni, et al., Molecules (2018) 23(10): 2478 and Chen, et al., Molecules (2016) 21:1709). Terpenes are a good candidate for CPE for their low toxicity and skin irritancy when compared to other commonly used synthetic CPEs. It is generally believed that terpenes aid skin penetration by increasing the stratum corneum partitioning of the drug.

Cannabinoids are metabolized within the body by enzymes from the cytochrome P450 (CYP450) family, such as CYP2C9 and CYP3A4. Antibiotics such as Clarithromycin and Erythromycin and certain antifungals from the azole class are inhibitors of said enzymes. The inhibition of these CYP450 enzymes slows down cannabinoid degradation and prolongs its pharmacologically active time. (Zendulka, et al., Curr Drug Metab (2016) 17(3):206-226). This allows for the therapeutic effect of a higher dose at a significantly lower dosage.

Although current commercially-available active hemostatic agents can provide immediate hemostasis for mild to moderate bleeding, they provide zero therapeutic properties nor aid in wound healing. There exists a need for an active hemostat that takes advantage of the various beneficial pharmacological and physiological properties of cannabinoids.

### SUMMARY OF THE INVENTION

The present invention relates to a wound management composition containing a full spectrum blend of active cannabinoids and at least one hemostatic agent including astringent-class hemostats, active-class hemostats, and active-mechanical-class hemostats. Further, the wound management composition may include at least chemical penetration enhancer that amplifies the absorption of cannabinoids, at least one local drug to prolong the pharmacologically active time of cannabinoids, at least one antibacterial agent, and at least one antifungal agent.

In embodiments, a wound management composition is disclosed including a full spectrum blend of active cannabinoids and at least one hemostatic agent containing astringent-class hemostats or active-class hemostats.

In one aspect, the astringent-class hemostats contain an active ingredient including aluminum sulfate, aluminum chloride, ferric sulfate, zinc chloride, epinephrine and combinations thereof. In a related aspect, the active-class hemostats contain an active ingredient including bovine thrombin, human-pooled thrombin, recombinant thrombin, fibrinogen and combinations thereof. In a further related aspect, the composition includes a chemical penetration enhancer that amplifies the absorption of cannabinoids.

In another related aspect, the composition includes an agent containing at least one antibacterial agent, at least one antifungal agent, and combinations thereof. In a further related aspect, the composition includes at least one local drug that prolongs the pharmacologically active time of the cannabinoids.

In one aspect, the full spectrum blend of active cannabinoids contains at least two cannabinoids including tetrahydrocannabinol (THC), cannabidiol (CBD), cannabinol (CBN), cannabichromene (CBC), cannabigerol (CBG), cannabivarin (CBV), tetrahydrocannabivarin (THCV) and cannabidivarin (CBDV), where the blend works in tandem to produce an interdependently enhancing entourage effect.

In one aspect, the concentration of cannabinoids in the wound management composition is greater than 0.3 wt. % in the composition.

In another aspect, the chemical penetration enhancer includes α-pinene, β-pinene, limonene, β-myrcene, β-caryophyllene, linalool, terpinolene, and oximene. In a related aspect, the concentration of the chemical penetration enhancer in the wound management composition is no less than 0.05 wt. % in the composition.

In one aspect, the antibacterial agent includes zinc oxide nanoparticles, silver nanoparticles, plant essential oils, clarithromycin, and erythromycin, and combinations thereof.

In another aspect, the antifungal agent includes fluconazole, itraconazole, ketoconazole, miconazole, and combinations thereof.

In one aspect, the local drug includes clarithromycin, erythromycin, fluconazole, itraconazole, ketoconazole, miconazole, and combinations thereof.

In embodiments, a method of delivery of the wound management composition is disclosed, including applying to a subject in need thereof a composition containing a wound management composition having a full spectrum blend of active cannabinoids and at least one hemostatic agent including astringent-class hemostats or active-class hemostats and a delivery device containing the wound management composition, where the delivery device includes a sponge, gauze, fabric-like mesh, a brush tip, a gel, a syringe, flour, a sheet, a solution, or a paste.

In one aspect, the wound management composition includes a full spectrum blend of active cannabinoids containing at least two cannabinoids including tetrahydrocannabinol (THC), cannabidiol (CBD), cannabinol (CBN), cannabichromene (CBC), cannabigerol (CBG), cannabivarin (CBV), tetrahydrocannabivarin (THCV), and cannabidivarin (CBDV).

In another aspect, the concentration of tetrahydrocannabinol in the wound management composition is greater than 0.3 wt. % in the composition.

In one aspect, the wound management composition further contains at least one hemostatic agent including a stringent-class hemostat or active-class hemostat.

In a related aspect, the stringent-class hemostats contain an active ingredient including aluminum sulfate, aluminum chloride, ferric sulfate, zinc chloride, epinephrine, and combinations thereof.

In another related aspect, the active-class hemostats contain an active ingredient including bovine thrombin, human-pooled thrombin, recombinant thrombin, fibrinogen, and combinations thereof.

In one aspect, the wound management composition further contains a chemical penetration enhancer including terpene, alpha-pinene, beta-pinene, limonene, beta-myrcene, beta caryophyllene, linalool, terpinolene, oximene, and combinations thereof.

In another aspect, the wound management composition further contains at least one antibacterial agent including zinc oxide nanoparticles, silver nanoparticles, plant essential oils, clarithromycin, and erythromycin, and combinations thereof.

In one aspect, the wound management composition further contains at least one antifungal agent including fluconazole, itraconazole, ketoconazole, miconazole, and combinations thereof.

In another aspect, the wound management composition further contains at least one local drug including clarithromycin, erythromycin, fluconazole, itraconazole, ketoconazole, miconazole, and combinations thereof.

In embodiments, a kit is disclosed having a full spectrum blend of active cannabinoids and at least one hemostatic agent including astringent-class hemostat or active-class hemostat: a sponge, gauze, fabric-like mesh, a brush tip, a gel, a syringe, flour, a sheet, a solution, or a paste a container comprising kit components: and instructions for application of said composition to a subject in need thereof.

In a related aspect, the full spectrum blend contains at least two cannabinoids including tetrahydrocannabinol (THC), cannabidiol (CBD), cannabinol (CBN), cannabichromene (CBC), cannabigerol (CBG), cannabivarin (CBV), tetrahydrocannabivarin (THCV), cannabidivarin (CBDV), and combinations thereof.

In one aspect, the kit contains stringent-class hemostats that have an active ingredient including aluminum sulfate, aluminum chloride, ferric sulfate, zinc chloride, epinephrine and combinations thereof or the kit contains active-class hemostats that have an active ingredient including bovine thrombin, human-pooled thrombin, recombinant thrombin, fibrinogen, and combinations thereof.

### BRIEF DESCRIPTION OF THE DRAWINGS

FIG. 1 illustrates the link between thrombin, vascular injury, coagulation, and platelet activation.

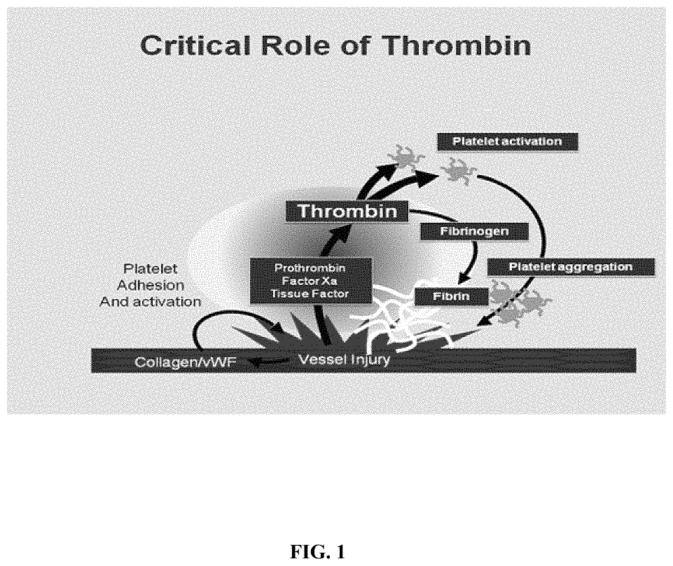

FIG. 2 is a scheme for decarboxylation and synthesis of the two principal cannabinoids: THC and CBD.

FIG. 3 shows groupings of various components of the endocannabinoid system.

FIG. 4 show the interaction between fibrinogen and thrombin to create fibrin-protofibril, a cluster of biopolymers grow into a fibril that initiates hemostasis within the body.

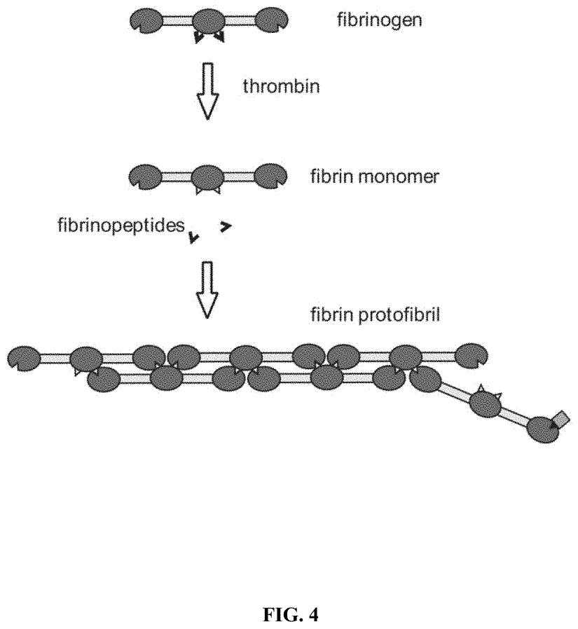

### DETAILED DESCRIPTION OF THE INVENTION

Before the present compositions, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a component” includes one or more components, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

As used herein, “about,” “approximately,” “substantially” and “significantly” will be understood by a person of ordinary skill in the art and will vary in some extent depending on the context in which they are used. If there are uses of the term which are not clear to persons of ordinary skill in the art given the context in which it is used, “about” and “approximately” will mean plus or minus <10% of a particular term and “substantially” and “significantly” will mean plus or minus >10% of the particular term.

The present invention relates to an active hemostatic composition comprising of a full spectrum blend of major cannabinoids.

The present invention seeks to improve on the prior art by disclosing a full-spectrum cannabinoid hemostat composition consisting of certain chemical penetration enhancers to aid absorption and CYP450 inhibitors to lengthen the active time of cannabinoids within the body. The present invention seeks to improve on the prior art by disclosing a full-spectrum cannabinoid topical composition consisting of certain chemical penetration enhancers to aid absorption and CYP450 inhibitors to lengthen the active time of cannabinoids within the body.

An oral care composition as disclosed herein includes a hemostatic agent. In certain embodiments, the hemostatic agent reduces the amount of gum bleeding by increasing the platelet aggregation in blood leading to hemostasis. An increase in hemostasis may be evidenced by a higher platelet aggregation area and/or a lower platelet rolling velocity. In certain embodiments, the hemostatic agent containing composition simultaneously provides an antibacterial effect. In some embodiments, the hemostatic agent is derived from or based upon compounds or extracts isolated from plants.

The present invention provides a novel therapeutic regimen for treating or preventing hypocoagulation and/or for preventing and treating bleeding in an individual in need thereof, comprising the administration of a wound management composition. Also, the invention provides pharmaceutical compositions which are administrable to a subject in need of enhanced blood coagulation wherein such need for enhanced blood coagulation may arise due to any bleeding disorder or condition, e.g., a genetic condition, e.g., a hemophilia, an acquired bleeding disorder, dysmenorrhea or an event such as an injury, trauma, or as a result of surgery, or an invasive procedure that may result in excessive bleeding.

The present invention relates to an oral hemostat composition comprising a full spectrum blend of major cannabinoids and at least one hemostatic agent containing astringent-class hemostats or active-class hemostats. Furthermore, the invention discloses mechanisms to enhance the delivery, absorption, and therapeutic effects of cannabinoids by the inclusion of various substances that will be detailed below.

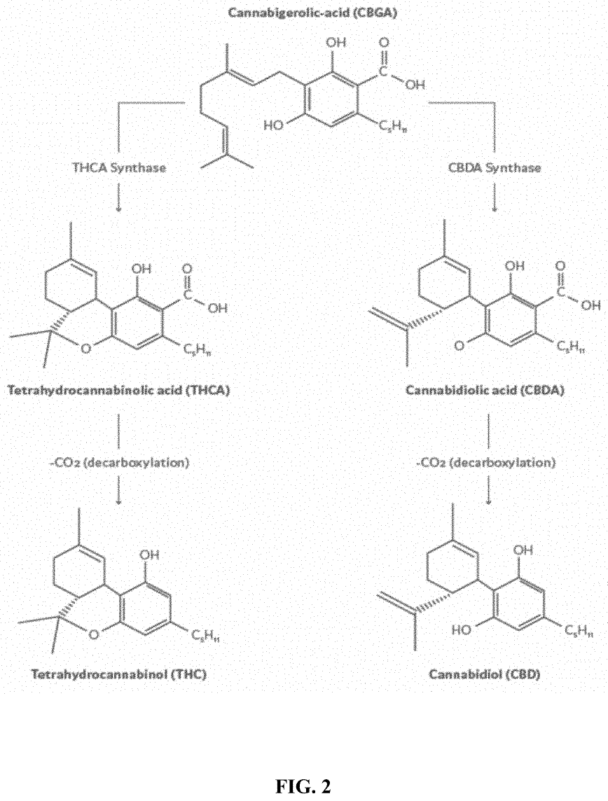

Within the scope of the present invention, a full-spectrum cannabinoid topical composition may include a combination of all 8 major cannabinoids: tetrahydrocannabinol (THC), cannabidiol (CBD), cannabinol (CBN), cannabichromene (CBC), cannabigerol (CBG), cannabivarin (CBV), tetrahydrocannabivarin (THCV), and cannabidivarin (CBDV). Although THC has desirable therapeutic benefits, its psychoactive effects are undesirable in a non-recreational environment. However, these adverse side effects can be mitigated by the inclusion of other major cannabinoids, specifically CBD, which can assuage some of the anxiety and forgetfulness produced by THC. Taken together, these cannabinoids work in tandem to create an interdependently enhancing entourage effect. The entourage effect is a synergistic mechanism by which the unwanted psychoactive effects of Cannabis are modulated, while its therapeutic effects are retained. Synthesis and structures of THC and CBD may be seen in FIG. 2.

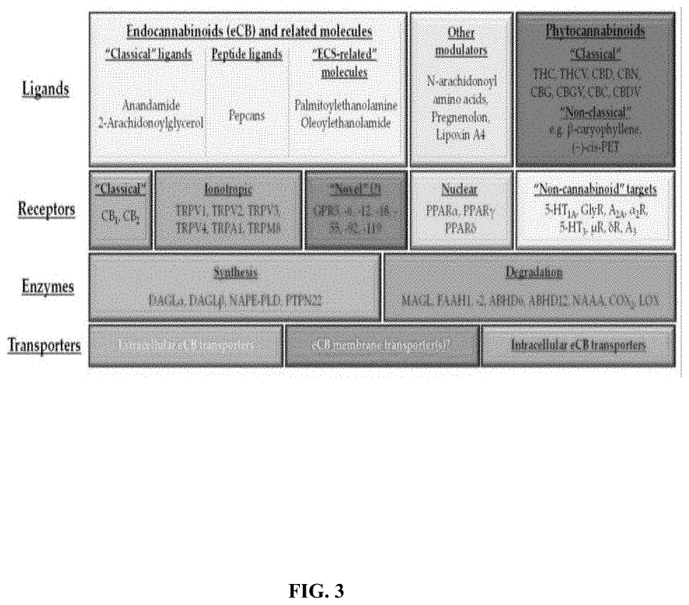

A cannabinoid is one of a class of diverse chemical compounds that acts on cannabinoid receptors, also known as the endocannabinoid system in cells that alter neurotransmitter release in the brain. Components of the endocannabinoid system may be seen in FIG. 3. Ligands for these receptor proteins include the endocannabinoids produced naturally in the body by animals; phytocannabinoids, found in cannabis; and synthetic cannabinoids, manufactured artificially. The most notable cannabinoid is the phytocannabinoid tetrahydrocannabinol (THC), the primary psychoactive compound in cannabis. Cannabidiol (CBD) is another major constituent of the plant.

The endo/phyto-cannabinoids include but are not limited to: N-acylethanolamindes which include N-arachidonoylethanolamide (better known as anandamide or more simply AEA), N-palmitoyl-ethanolamine (PEA), N-linoleoylethanolamide (LEA) and N-oleoylethanolamine (OEA).

Several classes of compounds with similarities in structure and/or activities to the THC purported active ingredient of the marijuana source plant have been identified. These are available in several plants outside the Cannabis genus and can be, cultured (e.g., through selective breeding or genetic engineering), extracted, purified or synthesized chemically de novo or from derivatives. Such compounds include, but are not limited to:

Cannabigerol class: cannabigerolic acid (CBGA) (antibiotic); cannabigerolic acid monomethylether (CBGAM); cannabigerol (CBG) (antibiotic, antifungal, anti-inflammatory, analgesic); Cannabigerol monomethylether (CBGM); cannabigerovarinic acid (CBGVA); Cannabigerovarin (CBGV).

Cannabichromene class: Cannabichromenic acid (CBCA); Cannabichromene (CBC) (antibiotic, antifungal, anti-inflammatory, analgesic); Cannabichromevarinic acid (CBCVA); Cannabichromevarin (CBCV); Cannabidiolic acid (CBDA) (antibiotic); Cannabidiol (CBD) ((antioxidant, anxiolytic, antispasmodic, anti-inflammatory, analgesic); cannabidiol monomethylether (CBDM); cannabidiol C4 (CBD-C4); cannabidivarinic acid (CBDVA); cannabidivarin (CBDV); cannabidiorcol (CBD-C1); Δ⁹-tetrahydrocannabinolic acid A (THCA-A); Δ⁹-tetrahydrocannabinolic acid B (THCA-B); 6a,10a-trans-6a,7,8,10a-tetrahydro-6,6,9-trimethyl-3-pentyl-6H-dibenzo[b,d]pyran-1-ol, (Δ⁹-tetrahydrocannabino-, THC) (analgesic, antioxidant, antiemetic, anti-inflammation); Δ⁹-tetrahydrocannabinolic acid-C4 (THCA-C4); Δ⁹-tetrahydrocannabinol-C4 (THC-C4); Δ⁹-tetrahydrocannabivarinic acid (THCVA); Δ⁹-tetrahydrocannabivarinic (THCV); Δ⁷-cis-isotetrahydrocannabivarin; Δ⁹-tetrahydro-cannabiorcolic acid (THCA-C1); tetrahydrocannabiorcol (THC-C1).

Δ⁸-tetrahydrocannabinol class: Δ⁸-tetrahydrocannabinolic acid (Δ⁸-TCA); Δ⁸-tetrahydrocannabinol (Δ⁸-THC).

Cannabicyclol class: cannabicyclol (CBL); cannabicyclolicacid (CBLA); cannabicyclovarin (CBLV).

Cannabieson class: cannabiesoic acid A (CBEA-A); cannabiesoic acid B (CBEA-B); cannabieson (CBE).

Cannabinol and cannabinodiol class: cannabinolic acid (CBNA); cannabinol (CBN); cannabinol methylether (CBNM); cannabinol-C4 (CBN-C4); cannabivarin (CBV); cannabinol-C2 (CBN-C2); cannabiorcol (CBN-C1); cannabinodiol (CBND); cannabinidivarin (CBDV).

Cannabitriol class: cannabitriol (CBT); 10-Ethoxy-9-hydroxy-Δ^{6a}tetrahydrocannabinol (10-EHDT); 8,9-dihydroxy-Δ^{6a}-tetrahydrocannabinol (8,9-DHDT); cannabitriolvarin (CBTV); ethoxy-cannabitriolvarin (CBTVE).

Miscellaneous class: dehydrocannabifuran (DCBF); cannabifuran (CBF); cannabichromanon (CBCN); cannabicitran (CBT); 10-oxo-Δ^{6a}-tetrahydrocannabinol (OTHC); Δ⁹-cis-tetrahydrocannabinol (cis-THC); 3,4,5,6-tetrahydro-7-hydroxy-α-α-2-trimethyl-9-n-propyl-2,6-m-ethano-2H-1-benzoxocin-5-methanol (2H-iso-HHCV); cannabiripsol (CBR); Trihydroxy-Δ⁹-tetrahydrocannabinol (triOH-THC).

LEA, PEA and OEA will bind to one or more of the endogenous cannabinoid receptors, but they are also important because they maintain AEA activity through their inhibition of the FAAH enzyme that is responsible for degrading AEA. N-alkylamides exert selective effects on the CB2, and have been shown to exert anti-inflammatory effects similar to AEA. Echinacea contains multiple N-alkylamides that have mimetic effects.

Phytoalkanes, another class of chemical compounds found in various plants, also have demonstrated cannabinolic modulation traits, e.g., N-alkanes ranging from C₉ to C₃₉, 2-methyl-, 3-methyl-, and some dimethyl alkanes are common in spices such as curcumin. The major alkane present in an essential oil obtained by extraction and steam distillation was the N-C29 alkane nonacosane (55.8 and 10.7%, respectively). Other abundant alkanes were heptacosane, 2,6-dimethyltetradecane, pentacosane, hexacosane, and hentriacontane. Curcumin reduces liver fibrosis by modulating cannabinoid receptor transmission.

The following list of compounds have been suggested to have cannabinolic activity (i.e., to bind and modulate activity of at least one human cannabinoid receptor): URB597, URB937, AM374, ARN2508, BIA 10-2474, BMS-469908, CAY-10402, JNJ-245, JNJ-1661010, JNJ-28833155, JNJ-40413269, JNJ-42119779, JNJ-42165279, LY-2183240, Cannabidiol, MK-3168, MK-4409, MM-433593, OL-92, OL-135, PF-622, PF-750, PF-3845, PF-04457845, PF-04862853, RN-450, SA-47, SA-73, SSR-411298, ST-4068, TK-25, URB524, URB597 (KDS-4103), URB694, URB937, VER-156084, V-158866, AM3506, AM6701, CAY10435, CAY10499, IDFP, JJKK-048, JNJ-40355003, JNJ-5003, JW618, JW651, JZL 184, JZL 195, JZP-372A, KML29, MAFP, MJN110,ML30, N-arachidonoyl maleimide, OL-135, OL92, PF-04457845, SA-57, ST4070, URB880, URB937, indomethacin, MK-886, resveratrol, cis-resveratrol, aspirin, COX-1 inhibitor II, loganin, tenidap, SC560, FR 122047 hydrochloride, valeryl salicylate, FR122047 hydrate, ibuprofen, TFAP, 6-methoxy-2-naphthylacetic acid, meloxicam, APHS, etodolac, meloxicam, meloxicam sodium salt, N-(4-acetamidophenyl)indomethacin amide, N-(2-phenylethyl)indomethacin amide, N-(3-pyridyl)indomethacin amide, indomethacin heptylester, SC236, sulinac, sulindac sulfide, pravadoline, naproxen, naproxen sodium salt, meclofenamate sodium, ibupropfen, S-ibuprofen, piroxicam, ketoprofen, S-ketoprofen, R-ibuprofen, ebselen, ETYA, diclofenac, diclofenac diethylamine, flurbiprofen, fexofenadine, pterostilbene, pterocarpus marsupium, 9,12-octadecadiynoic acid, ketorolac (tromethamine salt), NO-indomethacin, S-flurbiprofen, sedanolide, green tea extract (e.g., epicatechin), licofelone, lomoxicam, racibuprofen-d3, ampiroxicam, zaltoprofen, 7-(trifluoromethyl)1H-indole-2,3-dione, aceclofenac, acetylsalicylic acid-d4, S-ibuprofen lysinate, loxoprofen, CAY10589, ZU-6, isoicam, dipyrone, YS121 and MEG (mercaptoethylguanidine) and thus may be appropriate for use in the present invention.

In embodiments, a full spectrum blend of active cannabinoids refers to at least two cannabinoids including, but not limited to, tetrahydrocannabinol (THC), cannabidiol (CBD), cannabinol (CBN), cannabichromene (CBC), cannabigerol (CBG), cannabivarin (CBV), tetrahydrocannabivarin (THCV) and cannabidivarin (CBDV), which may work in tandem to produce an interdependently enhancing entourage effect.

Tetrahydrocannabinol (THC) is the principal psychoactive constituent of cannabis. With chemical name A-tetrahydrocannabinol, the term THC also refers to cannabinoid isomers. Recent research shows that THC exhibits potent anti-inflammatory and analgesic activity, is neuroprotective, and reduces intraocular pressure, spasticity, and muscle tension.

Cannabidiol (CBD) is a phytocannabinoid discovered in 1940. It is the most common cannabinoid produced by cannabis plants and accounts for up to 40% of the plant's extract. And because of the recent breeding efforts, CBD is becoming more commonly found. CBD exhibits analgesic and anti-inflammatory effects across a wide range of symptoms and conditions. CBD is also a very potent antioxidant. According to recent research, CBD has been shown to eliminate some of THC's unpleasant adverse effects, modulating its psychoactivity and reducing the incidence of THC-induced sedation, anxiety, and rapid heartbeat.

Cannabinol (CBN) is the oxidation byproduct of THC and among the more common cannabinoids found in the cannabis products. Studies on CBN have found that it may be a potent antibacterial agent. CBN may also be a powerful neuroprotectant and a potent anti-inflammatory agent. Further, a recent study indicated that CBN might be useful in treating burns because it reduces perceived thermal sensitivity.

Cannabichromene (CBC) has been shown to block pain and inflammation associated with collagen-induced osteoarthritis. Cannabinoids like CBC act on inflammation differently than non-steroidal anti-inflammatory drugs (NSAIDs), and don't have the side effects of these medications. In another example of the entourage effect, CBC in combination with THC had significant anti-inflammatory response in a recent animal study; together, the two cannabinoids produced a much greater effect on inflammation than by themselves.

CBG is Cannabigerol (CBG) is a non-psychoactive cannabinoid that plays an important role in the biochemistry of the cannabis plant. CBG acts as a chemical precursor to other cannabinoids such as THC and CBD. In animal experiments, CBG was found to be effective in decreasing the inflammation characteristics. European research shows evidence that CBG is an effective antibacterial agent, particularly against methicillin-resistant Staphylococcus aureus (MRSA) microbial strains resistant to several classes of drugs.

As disclosed herein, while not being bound by theory, full spectrum blend of these active cannabinoids works in tandem to produce an interdependently enhancing entourage effect. Further, the ratio of the hemostats (e.g., aluminum sulfate, aluminum chloride, ferric sulfate, zinc chloride, epinephrine, bovine thrombin, human-pooled thrombin, recombinant thrombin, fibrinogen and combinations thereof) and cannabinoids allows for an enhanced hemostatic effect.

In embodiments, the hemostats may be present at between about 15% to about 20% (w/v) oe about 20% to about 25% (w/v) relative to cannabinoid. In a related aspect, a composition may include aluminum sulfate, aluminum chloride, ferric sulfate, and/or zinc chloride at about, 15-25% w/v (alone or in combination); epinephrine at about 1:100,000 to about 1:50,000 (0.001-0.002% w/v); bovine, human and/or recombinant thrombin at about 500 IU/mL to about 1000 IU/mL or about 417 NIH/mL to about 833 NIH/mL (IU: International Standard unit for thrombin and NIH: US Standard unit for thrombin), and/or fibrinogen at about 2 mg/mL to about 4 mg/mL (about 0.2% to about 0.4% w/v), alone or in combination; cannabinoids (combined) at about 1% w/v to about 10% w/v; chemical penetration enhancers (combined) at about 0.05 w/v to about 5% w/v; anti-fungal agent at about 0.01% w/v to about 1% w/v; anti-bacterial agent at about 0.1% w/v to about 2% w/v; and local drug (e.g., clarithromycin, erythromycin, fluconazole, itraconazole, ketoconazole, miconazole) at about 0.1% w/v to about 2% w/v. In a related aspect, the composition as disclosed herein may comprise about 25% aluminum chloride, about 2% cannabinoids, and about 0.25% terpinolene. In another aspect, the composition may comprise about 15.5% ferric sulfate, about 3% cannabinoids, about 0.1% a-pinene, and about 0.1% β-pinene. In one aspect, the composition may comprise about 20% aluminum chloride, about 2% cannabinoids, about 0.5% limonene, and about 2% zinc oxide nanoparticles. In another aspect, the composition may comprise about 0.002% epinephrine in an 8% buffered dl-epinephrine hydrochloride solution, about 4% cannabinoids, about 0.2% myrcene, and about 1% clarithromycin. In one aspect, the composition may comprise about 500 IU/mL thrombin, about 0.2% fibrinogen, about 2% cannabinoids, about 0.5% oximene, and about 0.5% itraconazole. In a further related aspect, THC may be present at about 0.3% to about 0.4%, about 0.4% to about 0.5%, about 0.5% to about 1%, or about 1% to about 2% of the composition (w/v). As disclosed herein, all percentages are in w/v %.

The present invention further comprises certain local drugs, chemical penetration enhancers, antibacterial agent and antifungal agents, which will be disclosed in turn.

Certain drugs are incorporated to slow cannabinoids degradation and consequently lead to enhanced effects of cannabinoids and lengthen its pharmaceutically active time. In a related aspect, such drugs include, but are not limited to, amiodarone, clarithromycin, diltiazem, erythromycin, fluconazole, isoniazid, itraconazole, ketoconazole, miconazole, ritonavir, verapamil, and combinations thereof. In embodiments, the drugs may be present at about 0.1% to about 0.5%, about 0.5% to about 1.0%, about 1.0% to about 1.5%, or about 1.5% to about 2.0% w/v. In a related aspect, the drug concentration is about 0.1% to about 2.0% w/v.

Terpene, as a chemical penetration enhancer in the present invention, is selected from the group of α-pinene, β-pinene, limonene, β-myrcene, β-caryophyllene, linalool, terpinolene, oximene, and combinations thereof. Chemical penetration enhancers are agents that increase the transport of a drug across the skin barrier. They exert their effect by using a range of mechanisms such as disrupting the lipid bilayer structure in the stratum corneum (SC) and thereby increasing the drug's diffusion coefficient, extracting lipids from the SC, altering the solvent nature of the SC and consequently modifying the drug partitioning coefficient, acting on intracellular keratin, and the like. In a related aspect, terpene, as a chemical penetration may be used, which enhancer allows for better absorption of Cannabinoids and therefore provides enhanced effects and lengthened pharmaceutically active time of the wound management composition. In embodiments, the chemical penetration enhancer is about 0.05% to about 0.1%, about 0.1% to about 0.5%, about 0.5% to about 1.0%, about 1.0% to about 2%, about 2% to about 3%, about 3% to about 4%, or about 4% to about 5% w/v. In a related aspect, the enhancer is present at about 2% to about 5% w/v.

Antibacterial agents are a group of materials that fight against pathogenic bacteria. Thus, by killing or reducing the metabolic activity of bacteria, their pathogenic effect in the biological environments will be minimized. Furthermore, these materials can prevent bacterial plaque accumulation in the oral environment and therefore, can reduce the prevalence of plaque-related diseases such as caries. Antibacterial agents are included in the present invention to keep a healthy oral environment. In a related aspect, at least one antibacterial agent includes, but is not limited to, zinc oxide nanoparticles, silver nanoparticles, plant essential oils, clarithromycin, erythromycin, and combinations thereof. In embodiments, the agents may be present at about 0.1% to about 0.5%, about 0.5% to about 1.0%, about 1.0% to about 1.5%, or about 1.5% to about 2.0% w/v. In a related aspect, the drug concentration is about 0.1% to about 2.0% w/v.

An antifungal agent is a drug that selectively eliminates fungal pathogens from a host with minimal toxicity to the host. Antifungal agents are disclosed in the present invention to kill fungi or inhibit their growth for the benefits of the oral environment. In embodiments, at least one antifungal agent is selected from the group consisting of fluconazole, itraconazole, ketoconazole, miconazole, and combinations thereof. In embodiments, the antifungal agent is present at about 0.1% to about 0.2%, about 0.2% to about 0.5%, about 0.5% to about 0.7%, or about 0.7% to about 1.0% w/v.

The disclosed novel compositions are also suitable for formulating for standard oral applications because they can be combined with excipients including, but not limited to, flavorings, preservatives, and other active ingredients, including, but not limited to, nutrients, vitamins, omega-3 fatty acids, hyaluronic acid, disinfectants of the oral cavity, steroidal or non-steroidal anti-inflammatories, wound healing agents, analgesics, and antihistamines.

The composition of the invention may further comprise the usual adjuvants and/or additives such as preservatives/antioxidants, gels, fatty substances/oils, water, organic solvents, silicones, thickeners, softeners, emulsifiers, in addition to antifoaming agents, moisturizers, fragrances, sweeteners, surfactants, fillers, sequestering agents, anionic, cationic, nonionic or amphoteric polymers or mixtures thereof, propellants, acidifying or basifying agents, dyes, colorants, stabilizers, whitening agents, antibacterial agents, preservative active ingredients or any other ingredients usually formulated into drugs. The additives and/or additional active ingredients can, based on the desired product, easily be chosen by a person skilled in the art.

Physiologically acceptable carriers or excipients for use with the inventive pharmaceutical compositions can be routinely selected for a particular use by those skilled in the art. These include, but are not limited to, solvents, buffering agents, inert diluents or fillers, suspending agents, dispersing or wetting agents, preservatives, stabilizers, chelating agents, emulsifying agents, anti-foaming agents, gel-forming agents, ointment bases, humectants, emollients, and skin protecting agents.

Examples of solvents are water, alcohols, vegetable, marine and mineral oils, polyethylene glycols, propylene glycols, glycerol, and liquid polyalkylsiloxanes. Inert diluents or fillers may be sucrose, sorbitol, sugar, mannitol, microcrystalline cellulose, starches, calcium carbonate, sodium chloride, lactose, calcium phosphate, calcium sulfate, or sodium phosphate. Examples of buffering agents include citric acid, acetic acid, lactic acid, hydrogenophosphoric acid, and diethylamine. Suitable suspending agents are, for example, naturally occurring gums (e.g., acacia, arabic, xanthan, and tragacanth gum), celluloses (e.g., carboxymethyl-, hydroxyethyl-, hydroxypropyl-, and hydroxypropylmethyl-cellulose), alginates and chitosans. Examples of dispersing or wetting agents are naturally occurring phosphatides (e.g., lecithin or soybean lecithin), condensation products of ethylene oxide with fatty acids or with long chain aliphatic alcohols (e.g., polyoxyethylene stearate, polyoxyethylene sorbitol monooleate, and polyoxyethylene sorbitan monooleate).

Preservatives may be added to a composition of the invention to prevent microbial contamination that can affect the stability of the formulation and cause infection in the patient. Suitable examples of preservatives include parabens (such as methyl, ethyl, propyl, p-hydroxybenzoate, butyl, isobutyl, and isopropylparaben), potassium sorbate, sorbic acid, benzoic acid, methyl benzoate, phenoxyethanol, bronopol, bronidox, MDM hydantoin, iodopropynyl butylcarbamate, benzalconium chloride, cetrimide, and benzylalcohol. Examples of chelating agents include sodium EDTA and citric acid.

Examples of emulsifying agents are naturally occurring gums, naturally occurring phosphatides (e.g., soybean lecithin; sorbitan mono-oleate derivatives), sorbitan esters, monoglycerides, fatty alcohols, and fatty acid esters (e.g., triglycerides of fatty acids). Anti-foaming agents usually facilitate manufacture, they dissipate foam by destabilizing the air-liquid interface and allow liquid to drain away from air pockets. Examples of anti-foaming agents include simethicone, dimethicone, ethanol, and ether.

Generally, viscosity is an important parameter for oral care compositions, such as toothpastes or whitening gels. For example, when the viscosity of an oral care composition is too low, it may become too runny and physical phase separation may take place. In some cases, this will not only affect the aesthetics of the oral care composition but also the homogeneity of the ingredients in the oral care composition. On the other hand, if the viscosity of the oral care compositions is too high, the oral care composition will be difficult to manufacture and package.

In addition, oral care compositions with high viscosity are very difficult for users to evacuate from commonly used packages, such as tubes or syringes. Accordingly, it's important to select ingredients for oral care compositions that achieve a desirable range of viscosity to ensure product manufacturability, stability, and quality, as well as consumer acceptance.

Examples of viscosity-increasing agents are liquid paraffin, polyethylene, fatty oils, colloidal silica or aluminum, glycerol, propylene glycol, carboxyvinyl polymers, magnesium-aluminum silicates, hydrophilic polymers (such as, for example, starch or cellulose derivatives), water-swellable hydrocolloids, carrageenans, hyaluronates, and alginates. Ointment bases suitable for use in the compositions of the present invention may be hydrophobic or hydrophilic, and include paraffin, lanolin, liquid polyalkylsiloxanes, cetanol, cetyl palmitate, vegetable oils, sorbitan esters of fatty acids, polyethylene glycols, and condensation products between sorbitan esters of fatty acids, ethylene oxide (e.g., polyoxyethylene sorbitan monooleate), and polysorbates.

Examples of humectants are ethanol, isopropanol glycerin, propylene glycol, sorbitol, lactic acid, and urea. Suitable emollients include cholesterol and glycerol.

The compositions of the invention may, alternatively or additionally, comprise other types of excipients including, thickening agents and bioadhesive polymers.

Thickening agents are generally used to increase viscosity and improve bioadhesive properties of pharmaceutical compositions. Examples of thickening agents include, but are not limited to, celluloses, polyethylene glycol, polyethylene oxide, naturally occurring gums, gelatin, karaya, pectin, alginic acid, and povidone. Particularly interesting are thickening agents with thixotropic properties (i.e., agents whose viscosity is decreased by shaking or stirring). The presence of such an agent in a gel composition allows the viscosity of the composition to be reduced at the time of administration to facilitate its application to the site of interest (e.g., to the gingiva or periodontal pocket) and, to increase after application so that the composition remains at the site of administration.

The following examples will further describe the present invention without, however, at the same time, constituting any limitation thereof.

### EXAMPLES

### Example 1. Oral Hemostatic Gel with Cannabinoids

An oral hemostatic gel is prepared by mixing 15.5 ferric sulfate with cannabinoids selected from the group of THC, CBD, CBN, CBC, CBG, CBV, THCV and CBDV. Cannabinoids (combined) are included in the above formulations between 1-10% w/v.

### Example 2. Oral Hemostatic Solution with Cannabinoids

An oral hemostatic solution is prepared by mixing 25% buffered aluminum chloride with cannabinoids selected from the group of THC, CBD, CBN, CBC, CBG, CBV, THCV and CBDV. Cannabinoids (combined) are included in the above formulations between 1-10% w/v.

### Example 3. Topical Hemostatic Gel with Cannabinoids

A topical hemostatic gel is prepared by mixing 20% buffered aluminum sulfate with cannabinoids selected from the group of THC, CBD, CBN, CBC, CBG, CBV, THCV and CBDV. Cannabinoids (combined) are included in the above formulations between 1-10% w/v.

### Example 4. Topical Hemostatic Solution with Cannabinoids

A topical hemostatic solution is prepared by mixing 8% buffered dl-epinephrine hydrochloride with cannabinoids selected from the group of THC, CBD, CBN, CBC, CBG, CBV, THCV and CBDV. Cannabinoids (combined) are included in the above formulations between 1-10% w/v.

### Example 5. Oral Hemostatic Solution with Cannabinoids in Syringe

An oral hemostatic solution in a disposable syringe is prepared by mixing 750 IU/mL thrombin selected from the group of bovine thrombin, human-pooled thrombin, or recombinant thrombin with cannabinoids selected from the group of THC, CBD, CBN, CBC, CBG, CBV, THCV and CBDV. Cannabinoids (combined) are included in the above formulations between 1-10% w/v.

### Example 6. Topical Hemostatic Solution with Cannabinoids in Jar

A topical hemostatic solution in ajar is prepared by mixing 0.4% fibrinogen with cannabinoids selected from the group of THC, CBD, CBN, CBC, CBG, CBV, THCV and CBDV. Said solution is applied with a disposable cotton swab. Cannabinoids (combined) are included in the above formulations between 1-10% w/v.

### Example 7. Intraoral Hemostatic Gel with Cannabinoids in Syringe

An intraoral hemostatic gel in a needleless disposable syringe is prepared by mixing 500 IU/mL bovine thrombin, 0.2% fibrinogen, 0.5% miconazole, and cannabinoids selected from the group of THC, CBD, CBN, CBC, CBG, CBV, THCV and CBDV. Cannabinoids (combined) are included in the above formulations between 1-10% w/v.

### Example 8: Hemostatic Patch with Cannabinoids

A hemostatic patch is prepared by immersing then drying 500 IU/mL human-pooled thrombin, 0.2% fibrinogen, 0.5% itraconazole, and cannabinoids selected from the group of THC, CBD, CBN, CBC, CBG, CBV, THCV and CBDV in a biologically-absorbable patch selected from the group of fabric-like mesh, sponge or gauze. Said patch will be placed in a single-use, vacuum-sealed foil pouch to prevent UV light penetration and/or oxidation. Cannabinoids (combined) are included in the above formulations between 1-10% w/v.

Said jars and syringes are tinted in a dark color (e.g., brown) that may adequately block or filter out any UV light that may break down cannabinoids.

While a number of preferred embodiments of the invention and variations thereof have been described in detail, other modifications and methods of using and applications for the same will be apparent to those of skill in the art. Accordingly, it should be understood that various applications, modifications, materials, and substitutions may be made of equivalents without departing from the spirit of the invention or the scope of the claims. It should be understood that the invention is not limited to the embodiments set forth herein for purposes of exemplification, but is to be defined only by a fair reading of the appended claims, including the full range of equivalency to which each element thereof is entitled. All references recited herein are incorporated by reference herein in their entireties.

We claim herein:

1. A wound management composition comprising: a full spectrum blend of active cannabinoids and at least one hemostatic agent selected from astringent-class hemostats or active-class hemostats.

2. The wound management composition of claim 1, wherein said astringent-class hemostats contain an active ingredient selected from the group consisting of aluminum sulfate, aluminum chloride, ferric sulfate, zinc chloride, epinephrine, and combinations thereof.

3. The wound management composition of claim 1, wherein said active-class hemostats contain an active ingredient selected from the group consisting of bovine thrombin, human-pooled thrombin, recombinant thrombin, fibrinogen, and combinations thereof.

4. The wound management composition of claim 1, further comprising a chemical penetration enhancer that amplifies the absorption of cannabinoids.

5. The wound management composition of claim 1, further comprising an agent selected from the group consisting of at least one antibacterial agent, at least one antifungal agent, and combinations thereof.

6. The wound management composition of claim 1, further comprising at least one local drug that prolongs the pharmacologically active time of said cannabinoids.

7. The wound management composition of claim 1, wherein said full spectrum blend of active cannabinoids contain at least two cannabinoids selected from the group consisting of tetrahydrocannabinol (THC), cannabidiol (CBD), cannabinol (CBN), cannabichromene (CBC), cannabigerol (CBG), cannabivarin (CBV), tetrahydrocannabivarin (THCV), and cannabidivarin (CBDV), and wherein said blend works in tandem to produce an interdependently enhancing entourage effect.

8. The wound management composition of claim 1, wherein the concentration of cannabinoids in the wound management composition is greater than 0.3 wt. % in the composition.

9. The wound management composition of claim 4, wherein said chemical penetration enhancer consists of at least one terpene selected from the group of α-pinene, β-pinene, limonene, β-myrcene, β-caryophyllene, linalool, terpinolene, oximene, and combinations thereof.

10. The wound management composition of claim 9, wherein the concentration of said chemical penetration enhancer in the wound management composition is no less than 0.05 wt. % in the composition.

11. The wound management composition of claim 5, wherein said antibacterial agent is selected from the group consisting of zinc oxide nanoparticles, silver nanoparticles, plant essential oils, clarithromycin, and erythromycin, and combinations thereof.

12. The wound management composition of claim 5, wherein said antifungal agent is selected from the group consisting of fluconazole, itraconazole, ketoconazole, miconazole, and combinations thereof.

13. The wound management composition according to claim 6, wherein said local drug is selected from the group consisting of clarithromycin, erythromycin, fluconazole, itraconazole, ketoconazole, miconazole, and combinations thereof.

14. A method of delivery of a wound management composition comprising:
applying to a subject in need thereof a composition comprising:
(a) a wound management composition containing a full spectrum blend of active cannabinoids and at least one hemostatic agent selected from astringent-class hemostats or active-class hemostats; and
(b) a delivery device containing the wound management composition,
wherein the delivery device is selected from the group consisting of a sponge, gauze, fabric-like mesh, a brush tip, a syringe, flour, a sheet, a solution, or a paste.

15. The method of delivery of claim 14, wherein said wound management composition comprises a full spectrum blend of active cannabinoids containing at least two cannabinoids selected from the group consisting of tetrahydrocannabinol (THC), cannabidiol (CBD), cannabinol (CBN), cannabichromene (CBC), cannabigerol (CBG), cannabivarin (CBV), tetrahydrocannabivarin (THCV), cannabidivarin (CBDV), and combinations thereof.

16. The method of delivery of claim 14, wherein said concentration of tetrahydrocannabinol in the wound management composition is greater than 0.3 wt. % in the composition.

17. The method of delivery of claim 14, wherein said stringent-class hemostats contains an active ingredient selected from the group consisting of aluminum sulfate, aluminum chloride, ferric sulfate, zinc chloride, epinephrine, and combinations thereof.

18. The method of delivery of claim 14, wherein said active-class hemostats contains an active ingredient selected from the group consisting of bovine thrombin, human-pooled thrombin, recombinant thrombin, fibrinogen, and combinations thereof.

19. The method of delivery of claim 14, wherein said wound management composition further comprises a chemical penetration enhancer selected from the group consisting of terpene, α-pinene, β-pinene, limonene, β-myrcene, β-caryophyllene, linalool, terpinolene, oximene, and combinations thereof.

20. The method of delivery of claim 14, wherein said wound management composition further comprises at least one antibacterial agent selected from the group consisting of zinc oxide nanoparticles, silver nanoparticles, plant essential oils, clarithromycin, erythromycin, and combinations thereof.

21. The method of delivery of claim 14, wherein said wound management composition further comprises at least one antifungal agent selected from the group consisting of fluconazole, itraconazole, ketoconazole, miconazole, and combinations thereof.

22. The method of delivery of claim 14, wherein said wound management composition further comprises of at least one local drug selected from the group consisting of clarithromycin, erythromycin, fluconazole, itraconazole, ketoconazole, miconazole, and combinations thereof.

23. A kit comprising:
a) a full spectrum blend of active cannabinoids and at least one hemostatic agent selected from astringent-class hemostat or active-class hemostat;
b) a sponge, gauze, fabric-like mesh, a brush tip, a syringe, flour, a sheet, a solution, or a paste;
c) a container comprising kit components; and
d) instructions for mixing and applying (a) plus (b) to a subject in need thereof.

24. The kit of claim 23, wherein said full spectrum blend comprises at least two cannabinoids selected from the group consisting of tetrahydrocannabinol (THC), cannabidiol (CBD), cannabinol (CBN), cannabichromene (CBC), cannabigerol (CBG), cannabivarin (CBV), tetrahydrocannabivarin (THCV), cannabidivarin (CBDV), and combinations thereof.

25. The kit of claim 23, wherein the kit comprises stringent-class hemostats that contain an active ingredient selected from the group consisting of aluminum sulfate, aluminum chloride, ferric sulfate, zinc chloride, epinephrine, and combinations thereof or wherein the kit comprises active-class hemostats that contain an active ingredient selected from the group consisting of bovine thrombin, human-pooled thrombin, recombinant thrombin, fibrinogen, and combinations thereof.
